SEQUENCE LISTING (5 sequences)

SEQ ID NO: 1 - protein, 190 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCHCHMGTHCHSSDGPRGVIPEPRCP

SEQ ID NO: 2 - protein, 120 aa, Homo sapiens
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGT

SEQ ID NO: 3 - protein, 19 aa, Homo sapiens
VLLLRAGFYAVSFLSVAVG

SEQ ID NO: 4 - protein, 33 aa, Homo sapiens
STVYYQGKCHCHMGTHCHSSDGPRGVIPEPRCP

SEQ ID NO: 5 - protein, 15 aa, Artificial Sequence
  Description of Artificial Sequence peptide derived from natural sequence, useful for antiserum production
WVSQPPEIRTLEGSC

(19) United States
(12) Patent Application Publication
(10) Pub. No.: US 2008/0063717 A1
(43) Pub. Date: Mar. 13, 2008

(54) Pharmaceutical Compositions Having an Effect on the Proliferation of Nk Cells and a Method Using the Same

(75) Inventors: Francois Romagne, La Ciotat (FR); Andre Pascale, Marseille (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Innate Pharma, S.A.S., Marseille (FR)

(21) Appl. No.: 10/539,828

(22) PCT Filed: Dec. 22, 2003
(86) PCT No.: PCT/EP03/14716

§ 371 Date: Jul. 9, 2007

Related U.S. Application Data
(60) Provisional application No. 60/435,344, filed on Dec. 23, 2002.

(51) Int. Cl. A61K 39/395; A61K 38/20; A61K 9/14; A61P 43/00

(52) U.S. Cl. 424/489; 424/133.1; 424/139.1; 424/144.1; 424/173.1; 424/85.2

(57) ABSTRACT

The present invention relates to pharmaceutical compositions having an effect on the proliferation of NK cells, to a method for specifically stimulating the proliferation of NK cell and to the use of same in the manufacture of a drug for the antitumoral prevention, palliation, and therapy of e.g., melanoma, hepatocarcinoma or lung adenocarcinoma and for anti-microbial prevention, palliation and therapy.

### CROSS-REFERENCE TO RELATED APPLICATION

This application is the U.S. national stage application of International Patent Application No. PCT/EP2003/014716, filed Dec. 22, 2003, which claims the benefit of U.S. Patent Application No. 60/435,344, filed Dec. 23, 2002, the disclosures of which are hereby incorporated by reference in their entireties.

The invention relates to pharmaceutical compositions having an effect on the proliferation of NK cells, to a method for specifically stimulating the proliferation of NK cells and to the use of same in the manufacture of a drug for the antitumoral prevention, palliation, and therapy of e.g., melanoma, hepatocarcinoma or lung adenocarcinoma and for anti-microbial prevention, palliation and therapy.

Some mechanisms of cytotoxicity of NK cells are known for a long time.

NK cells express CD16 molecule, which is a low affinity receptor for the Fc portion of IgG molecules. Thus NK cells recognize and kill antibody coated targets through recognition of the Fc portion of antibodies, that specifically recognize structures on the target cells.

NK cells also express so called Killer Inhibitory Receptors (KIR), which specifically recognize MHC class I molecule and inhibit the activation of cytolytic pathway in NK cells. Thus MHC class I positive targets are protected to a certain level from NK cell lysis.

Nevertheless, some targets, that do not express MHC class I positive targets are not killed by NK cells. This suggested that an active mechanism, distinct of CD16 or KIR molecule, can activate NK cells.

Several NK specific receptors have been identified that play an important role in the activation of NK cells.

Thus, NKp46 has been disclosed as active receptor responsible for triggering the natural cytotoxicity. More recently, other triggering receptor involved in NK cell mediated recognition and killing of target cells have also been disclosed. Moretta et al have thus disclosed a receptor of about 30 kD on SDS PAGE, designated NKp30 (U.S. patent application Ser. No. 10/036,444 divisional of U.S. patent application Ser. No. 09/440,514).

Antibodies specific to these receptors, when coated to Fc receptor positive cells by their Fc moieties, trigger NK cell recognition and cytotoxicity in tests known as redirected killing assays.

It has been demonstrated that a lot of NK sensitive target are killed via one of these receptors, as Fab′2 or IgM specific for NkP46 or NKp30 abrogate most of the killing capacity of NK cells towards sensitive cells. This implies that specific ligands are present on sensitive cells for NKp46 and/or NKp30, though the molecular structure of these receptors have not been disclosed yet.

The transduction elements associated with NKp30 and NKp46 are FCeRIg and the zeta homodimer.

It was previously demonstrated that antibodies recognizing NKp30 and NKp46 could induce production of lymphokines by NK cells, and/or could induce cytotoxicity of NK cells in redirected killing assays.

The inventors demonstrate here that soluble anti NCR (NK Cell Receptor) antibodies can induce the specific proliferation of NK cells from fresh human PBMC, when used in association with cytokines. Interestingly, though CD16 shares the same transducing element (zeta homodimer and FceRIgamma), addition of soluble anti CD16 antibody did not support any specific increase of the NK cell population.

Moreover, as NKp30 and NKp46 are strictly restricted to NK cells, this demonstration gives the basis of a specific NK cell proliferation protocol.

It is thus an object of the invention to provide a pharmaceutical composition having, in particular, a stimulating effect on NK cell proliferation. It is another object of the invention to provide a method for specifically stimulating the proliferation of NK cells by using such a pharmaceutical composition.

The present invention relates also to the use of such a pharmaceutical composition in the manufacture of a drug for the prevention, palliation, and therapy of e.g., melanoma, hepatocarcinoma or lung adenocarcinoma and for anti-microbial prevention, palliation and therapy.

The pharmaceutical compositions of the invention comprise an effective amount of at least an antibody selected in the group comprising an anti-NCR antibody such as anti-NKp30 antibody or anti-NKp46 antibody, or both, or an immuno-reactive fragment thereof, and a cytokine selected in the group comprising interleukins such as IL2, IL12, IL15, IL21 or a combination thereof, in association with a pharmaceutically acceptable carrier, said antibody(ies) and cytokine(s) being administered together or separately to a subject. In a particular embodiment, the cytokine is IL2, IL15 or both. The pharmaceutical composition can comprise an expression vector encoding said cytokine. Said vector can be a viral vector and a plasmid vector. Alternatively, instead of administering said cytokine, the in vivo production of said cytokine can be induced.

In a preferred embodiment, anti-NKp30 and/or anti-NKp46 antibodies are used in admixture with IL2.

In said compositions, the anti-NKp30 antibodies are isolated antibodies or antigen binding fragments thereof which specifically bind to a polypeptide selected from the group consisting of SEQ ID No1, SEQ ID No2, SEQ ID No3, SEQ ID No4, or an immunogenic fragment thereof, and SEQ ID No5.

SEQ ID No1 relates to the human NKp30 190 aa polypeptide (about 30 kD on SDS-PAGE), which is selectively expressed by NK cells, and particularly mature NK cells; SEQ ID No2 relates to the extracellular region of human NKp30 receptor; SEQ ID No3 relates to the transmembrane region of human NKp30 receptor; SEQ ID No4 relates to the cytoplasmic tail of the human NKp30 receptor; SEQ ID No5 relates to a 15 aa immunogenic peptide derived from SEQ ID No1.

In said compositions, “anti-NKp46 antibodies” refer to isolated antibodies respectively against NK-p46.

Preferred antibodies specifically bind to polypeptide having SEQ ID No1.

The anti-NKp30 and/or anti-NKp46 antibodies of said compositions are advantageously monoclonal antibodies, affinity, chimerized or humanized antibodies and more preferably humanized mouse monoclonal antibodies or of human origin.

A more particularly preferred anti-NKp30 monoclonal antibody is produced by hybridoma strain I-2576.

In pharmaceutical compositions comprising immuno-reactive antibody fragments, said fragments are essentially Fab, F(ab′)₂, Fv fragments, and CDR grafted humanized antibody fragments.

The person skilled in the art will note that humanized antibodies of the invention can be derived therefrom as desired, notably when the pharmaceutical compositions according to the invention are intended to be administered to a human person. By [symbol] antibody immuno-reactive fragments>>, it is herein notably meant any antibody fragment comprising the antigen binding-site. Such fragments thus include F(ab′)₂ fragments obtained either by enzymatic digestion of said antibody by proteolytic enzymes such as pepsin or papaïn and Fab fragments derived thereof by reduction of sulfhydryl groups located in the hinge regions, as known by any skilled person. Immunoreactive fragments can also comprise recombinant single chain or dimeric polypeptides whose sequence comprises the CDR regions of the antibody of interest. Isolated CDR regions themselves are also contemplated within the definition of the isolated immuno-reactive fragments.

Said pharmaceutical compositions can be administered by various routes, including intradermal, intramuscular, intraperitoneal, intravenous, or subcutaneous injection, intranasal route and the chirurgical route.

Depending on the desired administration route, the galenic forms will be, for example, tablet, powder, pastes, patches, granules, microgranules, nanoparticules, colloid solution, aqueous solution, injectable solutions, sprays and liposomes. The galenic form may also correspond to slow and/or controlled release forms.

By [symbol] pharmaceutically acceptable vehicle [symbol] comprised in the pharmaceutical compositions of the invention it is meant herein a vehicle whose solubility and/or chemical and/or galenic properties are adapted to the desired administration route and the ailed efficiency level. Such vehicles may include saline or dextrose solutions. The pharmaceutical composition according to the invention may further comprise any appropriate buffer and/or stabilizing compound.

Generally speaking, the pharmaceutical compositions of the invention are useful in the pathologies susceptible to be controlled by NK cells.

Numerous cancer have been shown to be susceptible to NK cell lysis, i.e. melanoma, Chronic Myeloid Leukemia, Acute Myeloid Leukemia, Lymphomas, Multiple Myeloma, hepatocarcinoma, lung adenocarcinoma, Neuroblastoma . . . Virally infected cells are also susceptible to NK cell lysis such as CMV, EBV, HIV, HCV etc.

The pharmaceutical compositions of the inventions are particularly useful for anti-tumoral prevention, palliation, therapy e.g., of melanoma, hepatocarcinoma or lung adenocarcinoma and for anti-microbial prevention, palliation and therapy.

The dosage will be chosen depending on the condition of the patient to be treated.

An effective dose typically ranges from 1 ng to 100 mg/kg (body weight) of anti-NCR antibodies, and typically lower than 1 million units/square meters/day of cytokine(s), when the pharmaceutical composition of the invention is used for daily subcutaneous injection. In fact, the amount of anti-NCR antibody to be used in such an in vivo pharmaceutical composition of the invention to obtain a specific proliferation of NK cells will notably depend on the particular antibody or antibodies used (affinity, chimerized or humanized antibody). The antibody should preferably be used to obtain an effective concentration for stimulation, without inducing a depletion of the NK cells or toxicity.

Advantageously, said interleukine is IL-2 and is injected subcutaneously at daily doses below 1 million units/m² for 5 to 10 days.

The invention also relates to a method for stimulating the proliferation of NK cells which comprises contacting NK cells with an effective amount of a pharmaceutical composition as above defined.

Advantageously, the method of the invention comprises one or several injections of an effective amount of at least an antibody selected in the group comprising an anti-NCR antibody such as anti-NKp30 antibody or anti-NKp46 antibody, or both, or an immuno-reactive fragment thereof, and, repeated injections of a cytokine selected in the group comprising interleukins such as IL2 (Research Diagnostics, NJ, RDI-202), IL12 (Research Diagnostics, NJ, DI-212), IL15 (Research Diagnostics, NJ, RDI-215), IL21 (Asano et al, FEBS Lett. 2002; 528:70-6) or a combination thereof, during 5-10 days, said cytokine(s) being first injected on the same day as the first injection of antibodies. Preferably, the cytokine is IL2, IL15 or both.

Said method preferably comprises one or two injections/day of cytokine(s) by subcutaneous route.

The invention also relates to the use of said pharmaceutical composition in the manufacture of a drug for the antitumoral prevention, palliation, and therapy of e.g., melanoma, Chronic Myeloid Leukemia, Acute Myeloid Leukemia, Lymphomas, Multiple Myeloma, hepatocarcinoma, lung adenocarcinoma, Neuroblastoma and for microbial prevention, palliation an therapy.

These and other features and advantages of the invention will be further apparent from the following examples. These examples are given for illustrative purposes only, and are in no way intended to restrict the scope of the present invention. Alternatives embodiments intended by any skilled person are encompassed by the present invention.

### DESCRIPTION OF THE DRAWINGS

In these examples, reference is made to FIGS. 1 to 6.

FIG. 1: Peripheral blood mononuclear cells (PBMC) from one healthy donor was cultivated with indicated antibodies (AZ20=anti NKp30, Bab281=anti NKp46), at 10 or 30 μg/ml in the presence of 50 units/ml IL2 until day 6, and either 50 units/ml (black bars) or 400 units/ml green bars) from day 6 to day 10. % of NK cells was determined by flow cytometry at day 10.

FIG. 2: Relative fold increase of NK (% NK cell at indicated day divided by % NK cells at day 0) from total unfractioned PBMC from 4 healthy donors with 10 μg/ml indicated antibodies at start and 50 units/ml IL2 along culture. Mean of relative fold increase, +/− standard deviations are represented.

FIG. 3: Carboxyfluorescein succinimidyl ester (CFSE) labelling (FL1, log scale, X axis) of NK cells (gated on CD56+/CD3− cells after 6 days of culture with the indicated treatment.

FIG. 4. AZ20 combined with IL-2 or IL-15 induce NK cells expansion. Freshly isolated PBMC were cultured under different conditions of interleukins (from day 0 to day 6: concentration is the bottom one; from day 6 to day 13: concentration of interleukin is the upper one) and with either an anti-CD56 mAb (N901, IgG1, 10 μg/ml) or an anti-NKp30 mAb (AZ20, IgG1, 10 μg/ml). At day 13 cells were collected and analyzed by flow cytometry for the % of NK cells defined as CD56+CD3− lymphocytes.

FIG. 5: Freshly isolated PBMC from 3 donors (A, B, C) were cultured with the indicated amount of AZ20 in RPMI 1640 10% FCS containing IL-2 (50 u/ml from day 0 to 6 and 400 u/ml from day 6) and IL-15 (10 ng/ml). At day 13 cells were collected; viability and count were assessed by trypan blue and % CD56⁺CD3⁻ lymphocytes by flow cytometry.

FIG. 6: CD25 induction of NK cells obtained after 6 days of stimulation of PBMC of two healthy donors (see material and methods), with indicated stimulus (IL2 (50 U/ml), mAbs 10 μg/ml).

### 1. MATERIALS AND METHODS

### Materials

.Blood:

For the first experiments, peripheral blood (5 to 10×7 ml EDTA-tubes, Becton Dickinson #367655) was collected from healthy volunteers (Lab. Hématologie, La Conception) and processed within two hours.

For further experiments, peripheral blood samples were provided by Etablissement Français du Sang (EFS) and processed within 24 hours (the blood is collected in bag containing 63 ml of anticoagulant CPD for the collect of 450 ml±10% blood; Baxter #R8443).

.PBMC and Primary Cell Culture:

50 ml polypropylene conical tubes (Falcon, #35 2070).

96 well plate U form (Falcon, #35 7525)

RPMI 1640 medium (Invitrogen, #31870074)

Fetal calf Serum (Invitrogen, #10270-106, Lot #40A0285K) heat inactivated

Penicillin-Streptomycin (5000 u/ml Invitrogen, #15070071)

Sodium Pyruvate (100 mM, Invitrogen, #11360088)

L-Glutamine 200 mM (100×, Invitrogen, #25030123)

Ficoll-Paque™ PLUS (Amersham Pharmacia Biotech, #17-1440-03)

Trypan Blue 0.4% (Invitrogen, #15250061)

D-PBS (1×) (Invitrogen, #14190169)

Hemacytometer (Neubauer)

Human recombinant IL-15 (25 μg, R&D, #219-IL-025). Stock solution of IL-15 (10 μg/ml) was prepared in PBS/BSA 0.1%, aliquoted and stored at −20° C.

Human recombinant IL-2 (Proleukin, 18×10⁶ IU, batch A199606/2, Chiron). Stock solutions of IL-2 (2×10⁶ and 2×10⁵ u/ml) were prepared in PBS/BSA 0.2% aliquoted and stored at −20° C.

Monoclonal Antibodies:
3G8 (anti-CD16), 5 mg/ ml, Beckman Coulter Immunotech.
N901 (anti-CD56), 5 mg/ml, Beckman Coulter Immunotech.
   Bab281 (anti-NKp46), 2.8 mg/ml (mAb purified on protein A Sepharose® from mice ascites)
AZ20 (anti-NKp30), 1 mg/ml and 1.2 mg/ml (mAb purified on protein A sepharose from mice ascites).

.Cell Division Analysis (CFSE Labelling):

5-(and 6)-carboxytetramethylrhodamine, succinimidyl ester (5(6)-TAMRA, SE) mixed isomers (CFSE, 25 mg; Molecular Probes, C-1157)

DMSO hybri-Max® (Sigma #D 2650)

Stock solution of CFSE (10 mM) in DMSO was prepared, aliquoted and stored at −20° C. as described in the technical data sheet provided by Molecular Probes.

.Staining:

96 well plate U form (Greiner #650 180)

1.2 ml micro titer tubes (QSP, #845-F)

5 ml tubes (12×75 PRO, CML, #TH5-12PRO)

Staining Buffer: PBS/0.2% BSA/0.02% Sodium Azide (D-PBS (10×), Invitrogen #14200083; Albumin Bovine, Fraction V, Invitrogen #; Sodium azide, Prolabo #27 967.150)

Mouse Serum NMRI (Janvier)

Commercialy available Ab used in this study (Table 1)

.Flow Cytometry:

Samples were run on a XL/MCL cytometer (Beckman Coulter). Acquisition and analysis were performed with EXPO™ 32 v1.2 software (Beckman Coulter).

### Methods:

.Preparation of PBMC:

Blood samples were diluted volume/volume with RPMI and processed using a classical ficoll procedure.

PBMC were collected in 50 ml conical tubes, washed 4 times with RPMI, 2% FCS, counted with trypan blue. Cells were resuspended at 2*10EE6 cells per ml in complete medium (RPMI 1640, PCS 10%, PS (50 u/ml), Glu 2 mM, Na. Pyr. 1 mM) for the initiation of cell culture, or 10EE7 cells/ml in staining buffer (PBS, 0.2% BSA, 0.02% Sodium Azide) for flow cytometry experiments.

.CFSE Labelling:

PBMC (10⁷cells/ml) were incubated 10 to 25 minutes at 37° C. (Water Bath) in RPMI/FCS2% containing CFSE (5 to 10 μM).

Cells were washed 3 times (10 min, 1200 RPM) with large volumes of cold (4° C.) RPMI/FCS 2%.

PBMC were resuspended in complete medium (2×10⁶ cells/ml) and were ready for cell culture.

For each of the subsequent technique, inventors recommend the following steps.

.Primary Cell Culture:

Day 0

Resuspend PBMC (2×10⁶/ml) in complete medium (RPMI 1640, FCS 10%, PS (50 u/ml), Glu 2 mM, Na. Pyr. 1 mM).

Prepare 2× interleukin stocks (IL-2, IL-15 and IL-2+IL-15) with complete medium.

Depending of the experiment, IL-2 was used at 50 or 400 u/ml final. IL-15 was always used at 10 ng/ml final.

Prepare 4× antibody stocks with complete medium.

Set up the culture: 50 μl 4× mAb+100 μl 2× interleukine+50 μl PBMC (10⁵ cells/well)

fill up to 200 μl with complete medium.

Day 3:

Change medium: remove 100 μl and add 100 μl complete medium containing 1× interleukin.

Day 6:

Change medium: remove 100 μl and add 100 μl complete medium containing either 50 u/ml IL-2 (±IL-15 10 ng/ml) or 400 u/ml IL-2 (±IL-15 10 ng/ml).

Day 9:

Split cells ½ and add medium (day 6)

Day 13, 16 and 20:

same as day 6 or day 9 depending of the cell growth.

.Staining:

Volumes, dilutions and Ab concentrations used in this study are indicated in Table 1.

For staining of cultured PBMC, 1 or 2 wells might be used for 1 point of staining. Control samples were prepared with interleukin stimulated cells.

% NK cells (defined as CD56⁺CD3⁻ cells) were checked at day 0, day 6, day 13, day 16 and day 20 and for some experiments at day 3, day 9 and day 35.

Characterization of the NK cell and T lymphocyte compartments at day 0 and day 17 (for some experiments) with antibodies listed in Table 1

Distribute mAb and adjust volume to 50 μl with staining buffer.

Add 50 μl of cell suspension.

Incubate 30 mns on ice.

Wash two times with staining buffer.

Resuspend cells in 150 μl of staining buffer and transfer to RT15 tubes containing 150 μl of staining buffer.

Keep refrigerated until acquisition on flow cytometer.

.Cytometry:

### Acquisition:

Run the isotype control mix in “set up mode” and set up: FSC, SSC, Threshold, FL1, FL2, FL3 and FL4 parameters:
Analysis was focused on lymphocytes identified by their FSC and SSC features (FSC, linear, Gain: 2, Volts: 400 and SSC, linear, Gain: 20, Volts: 400; Threshold: FSC, 150); the volts of these parameters might slightly differ analyse each experiment of this study (the FSC and SSC of cultured cells are usually higher than those of freshly isolated cells).
Draw the lymphocyte gate=Ly (acquire at least 10 000 events in Ly but all the events are collected)
Set up the volts for each fluorescent probe used in the experiment (aproximatively, FL1=800; FL2=800; FL3=950 and FL4=1000); they might slightly differ between each experiment).

Set up the compensations using single staining sample (mAbs used in the experiment or anti-CD8):
first, run FL1-mAb sample and set up FL2-FL1 (=15-20) in order to have the same FL2-MFI for the FL1-negative and the FL1-positive cells and all the FL2-negative cells in the first decade (<0.5% in the FL2 histogram and in FL1/FL2 dot plot); then, set up FL3-FL1 and FL4-FL1 as just described for FL2-FL1. Write the values and clear the compensations.
Repeat this step for each fluorescent probe.
Copy all the values to the compensation matrix.

Acquire the isotypic control sample and then, all the samples prepared for the experiment (write the lmd number corresponding to the acquired sample on the “96 well table”).

Each sample (lmd) is recorded and then transfert in a folder called: year, month, day (for example: 20020126). This folder is located in the HC/PA folder.

Acquisition of CFSE Samples:
FL1 compensations must be done using CFSE labelled cells only.
First set up the volts for FL1, FL2, . . . with the isotypic control sample; then, run the CFSE sample. The labelling is good when all the cells are positive for CFSE, the staining homogeneous and the pic channel located in the middle of the last decade of the FL1 histogram (without lowering the FL1 volts).
Set up all the compensations (they are usually higher than those obtained with FL1-mAb stained cells).

### Analysis:

Analysis was focused on lymphocytes identified by their FSC and SSC features (dot plot FSC/SSC). Draw the lymphocyte gate (Ly).

Quadrant regions (for dot plot) and marker regions (for histogram) were set with isotypic control samples (for all the fluorescences: % FLX⁺ cells<0.5%)

Analysis of the T cell or NK cell compartments: T cells=CD3⁺ lymphocytes were defined as the positive cells of the anti-CD3 staining histogram gated on Ly.

NK cells=CD3⁻-CD56⁺ lymphocytes corresponds to the CD3⁻-CD56⁺ gate in the CD3/CD56 dot plot (upper left part of the quadrant).

CFSE staining, CD25 expression (%), NKR expression (%) and CD56 density (MFI) were analysed.

.Cell Count and Freezing:

Some cultures were checked for cell numbers and cell viability (Trypan blue exclusion) and then frozen at the end of the experiment (day 20 or 35).

### Voir les Commentaires à Donner pour les Colonnes 2, 5 et 6

TABLE 1
Antibodies and reagents for cell cytometry.

| Specificity | Clone | Isotype | Cond. | Origin | Cat.# | Vol./Conc./dilution | |
|---|---|---|---|---|---|---|---|
| CD3 | UCHT1 | mIgG1 | FITC | BC Iot | IM1281 | 5 | μl |
| CD3 | UCHT1 | mIgG1 | PE | BC Iot | IM1282 | 5 | μl |
| CD3 | UCHT1 | mIgG1 | PC5 | BC Iot | IM2635 | 5 | μl |
| CD3 | UCHT1 | mIgG1 | ECD | BC Iot | IM2705 | 5 | μl |
| CD8 | B9.11 | mIgG1 | FITC | BC Iot | IM0451 | 5 | μl |
| CD8 | B9.11 | mIgG1 | PE | BC Iot | IM0452 | 5 | μl |
| CD8 | B9.11 | mIgG1 | PC5 | BC Iot | IM2638 | 5 | μl |
| CD8 | SFCI21ThyD3 | mIgG1 | ECD | BC | 6607011 | 5 | μl |
| CD16 | 3G8 | mIgG1 | purified | BC Iot | 813 | | |
| CD16 | 3G8 | mIgG1 | PE | BC Iot | IM1238 | 5 | μl |
| CD16 | 3G8 | mIgG1 | FITC | BC Iot | IM0814 | 5 | μl |
| CD25 | B1.49.9 | mIgG2a | FITC | BC Iot | IM0478 | 10 | μl |
| CD25 | B1.49.9 | mIgG2a | PC5 | BC Iot | IM2646 | 5 | μl |
| CD25 | M-A251 | mIgG1,k | PE | BD | 555432 | 5 | μl |
| CD27 | 1A4-CD27 | mIgG1,k | PE | BC Iot | 2578 | 5 | μl |
| CD45 | IMMU 19.2 | mIgG1 | PC5 | BC Iot | IM2652 | 5 | μl |
| CD54 | 84H10 | mIgG1 | PE | BC Iot | IM1239 | 5 | μl |
| CD56 | N901 (NKH-1) | mIgG1 | purified | BC Iot | 6602705 | | |
| CD56 | N901 (NKH-1) | mIgG1 | PE | BC Iot | IM2073 | 5 | μl |
| CD56 | N901 (NKH-1) | mIgG1 | PC5 | BC Iot | IM2654 | 3 | μl |
| CD57 | NC1 | mIgM | PE | BC Iot | IM2377 | 5 | μl |
| CD62L | DREG56 | mIgG1 | PE | BC Iot | IM2214 | 5 | μl |
| CD69 | TP1.55.3 | mIgG2b | PE | BC Iot | IM1943 | 4 | μl |
| CD94 | HP-3B1 | mIgG2a | PE | BC Iot | IM2276 | 5 | μl |
| CD122 | CF1 | mIgG1 | PE | BC Iot | IM1978 | 5 | μl |
| CD158a | EB6 | mIgG1 | PE | BC Iot | IM2277 | 10 | μl |
| CD158b | GL183 | mIgG1 | PE | BC Iot | IM2278 | 5 | μl |
| CD158e1/e2 | Z27 | mIgG1 | PE | BC Iot | IM3292 | 5 | μl |
| CD158i | FES172 | mIgG2a | PE | BC Iot | IM3337 | 5 | μl |
| CD158k | Q66 | mIgM | ascite | BC Iot | | 1:2000 | |
| CD159a | Z199 | mIgG2b | PE | BC Iot | IM3291 | 5 | μl |
| CD161 | 191B8 | mIgG2a | PE | BC Iot | IM3450 | 5 | μl |
| CD162R | 5H10 | mIgM | biotin | IP (AT, HC) | | 1 | μg/ml |
| CD244 | C1.7.1 | mIgG1 | PE | BC Iot | IM1608 | 5 | μl |
| Isotype control | 679.1Mc7 | mIgG1 | purified | BC Iot | IM0571 | 10 | μl |
| Isotype control | 679.1Mc7 | mIgG1 | ECD | BC Iot | IM2714 | 5 | μl |
| Isotype control | 679.1Mc7 | mIgG1 | FITC | BC Iot | IM0639 | 5 | μl |
| Isotype control | 679.1Mc7 | mIgG1 | PE | BC Iot | IM0670 | 5 | μl |
| Isotype control | 679.1Mc7 | mIgG1 | PC5 | BC Iot | IM2663 | 3 | μl |
| Isotype control | U7.27 | mIgG2a | PE | BC Iot | IM0671 | 5 | μl |
| Isotype control | MOPC-195 | mIgG2b | RD1 | BC Iot | 6603038 | 5 | μl |
| Isotype control | GC323 | mIgM | purified | BC Iot | IM1268 | 10 | μl |
| Isotype control | GC323 | mIgM | RD1 | BC Iot | 6602940 | 5 | μl |

### EXAMPLE 1

### Anti NC.R Antibodies+IL2 Can Promote Specific Cell Proliferation of NK Cells

### Relative Amplification of NK Cells after Stimulation with Anti NCR Antibodies and IL2.

PBMC from one donor has been isolated and tested for their in vitro response to combination of IL2 with either CD16, NKp30, NKp46 or CD56 mAbs. Cells were treated as described in material and methods, in the presence of saturating amount of antibodies.

Cells were monitored by flow cytometry and relative percentage of CD56+/CD3− (NK cells) was determined.

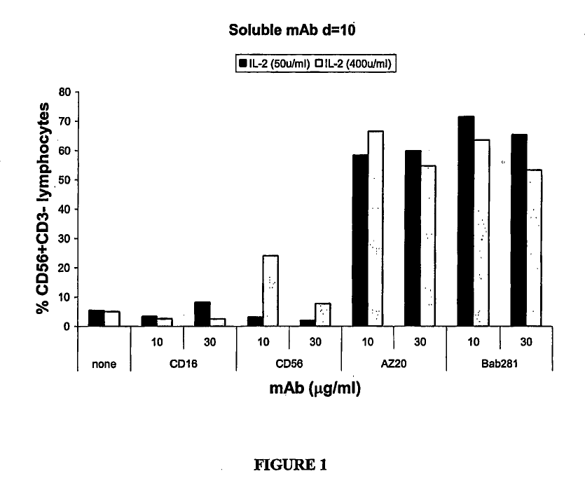

The results are presented in FIG. 1.

For this donor, there was an enrichment in NK cells in the culture at day 10 in the presence of anti NCR antibodies, whereas CD16 or CD56 induced no significant enrichment relative to IL2 alone.

To evaluate if this enrichment is donor related or not, PBMC have been isolated from 4 healthy volunteers and tested for their in vitro response to combination of IL2+ monoclonal antibodies against NCR.

Cells were treated as described in material and methods and put in the presence of saturating amounts (10 μg/ml) of either no antibodies, anti-NKp30, anti-NKp46, combination of NKp30 and NKp46, anti-CD56 monoclonal antibodies. Cells were monitored by flow cytometry and relative percentage of CD56+/CD3− (NK cells) was determined.

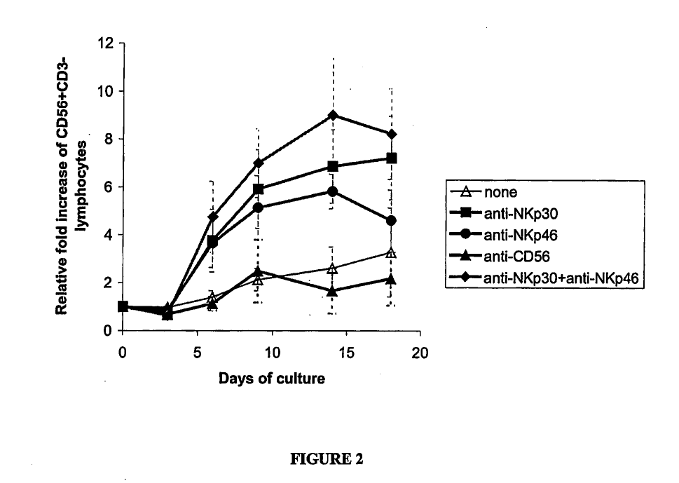

The results are presented in FIG. 2.

For the four healthy donors tested, there was a selective enrichment in NK cells. The enrichment is slightly better when anti NKp30 is used as compared to anti NKp46. The combination of the two antibodies gives the best enrichment.

The conclusion of these two studies is that the combination of anti NCR antibodies, with low dosage of IL2 (50 units/ml), induces a selective enrichment of NK cells.

To evaluate if this expansion is due to effective proliferation of NK cells or to selective death of the other cells present in the culture, PBMC were stained with CFSE, then washed to get rid of excess dye, at the initiation of the culture (see material and methods). CFSE is a stable fluorescent label that attach covalently to the cells. When the cells divide, about half of the initial dye content is present on the two daughter cells. If cells divide again, ¼^{th} of the initial dye content is present on the 4 daughter cells etc. Labelled cells were put in culture and stimulated by anti NCR antibodies and IL2 as above. Dye content of the cells is monitored by flow cytometry.

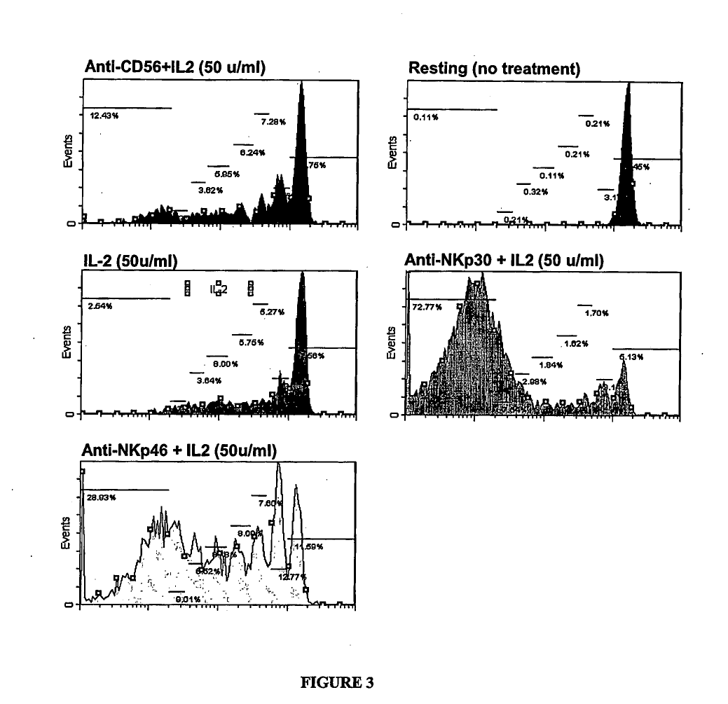

The result obtained on one representative donor is given in FIG. 3.

NKp30 or NKp46+IL2 co-treatment induces a better proliferation of NK cells than IL2 alone or IL2+irrelevant mAb (CD56) as indicated by the numbers of cells remaining with fluorescence intensity equivalent to resting cells (no division): 50 and 40% for IL2 and IL2+CD56 respectively, and 5 and 11% for NKp30+IL2 and NKp46+IL2 respectively.

The best proliferation was obtained for NKp30 where more than 80% of the cells in the culture at day 6 underwent more than 5 divisions.

To conclude, anti NCR (NKp30, NKp46 or both)+IL2 co-treatment induce selective proliferation of NK cells from PBMC in vitro.

### EXAMPLE 2

### Anti NCR+IL15 also Induces the Specific Expansion of NK Cells

The presence of a cytokine is crucial to sustain the expansion of the cells, after stimulation with the antibody. Experiments were carried out for testing if IL15 could also sustain the expansion of the cells on one donor.

Cells were stimulated with anti NKp30, and cultured in the presence of IL2, IL15 or both.

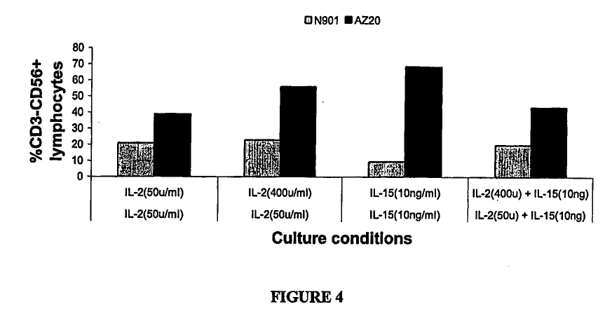

The results are presented in FIG. 4.

On this donor, IL15 was able to sustain the proliferation of NK cells.

We checked also that the combination of IL2 and IL15 also sustain the proliferation of NK cells. In other experiments on other donors, it was observed that the combination of IL2 and IL15 can be slightly better than the two cytokines alone.

### EXAMPLE 3

### Titration Curve of Anti NCR Antibodies for Induction of Proliferation

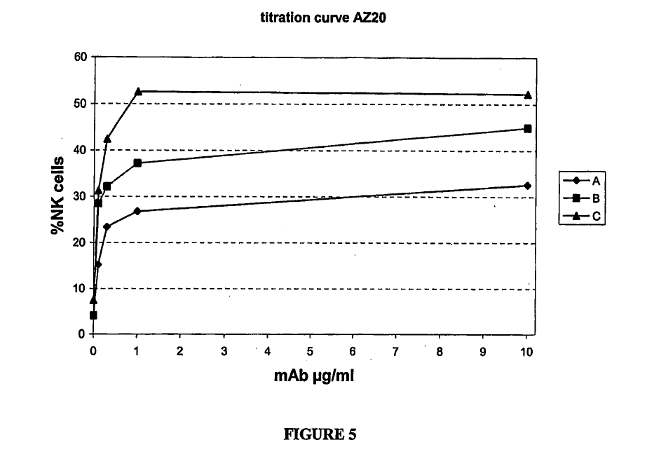

To evaluate the amount of antibody necessary to obtain a proliferation, a titration curve has been established with 3 independent donors with anti NKp30 antibody. The results are shown in FIG. 5. This experiment shows that the effect of the antibody is saturable with a plateau effect at about 1 μg/ml. The dose to obtain 50% of maximum effect is below 0.1 μg/ml in this experiment.

It should be noted that the characteristics of the curve may depend on the particular antibody used, and particularly of its affinity. The use of humanized anti NCR antibodies may also display a different titration curve.

### EXAMPLE 4

### Conditions of Use of Anti NCR Antibodies+IL2 In Vivo

The anti-NCR antibody or antibodies were tested first in vitro, and then in a relevant animal model.

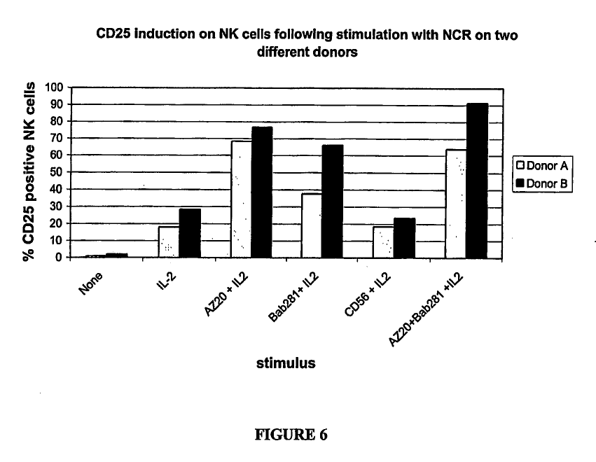

It should be noted that anti NCR+IL2 in vitro induces CD25 (FIG. 6), and thus the high affinity receptor for IL2 on most NK cells. In vitro, low doses such as 50 units/ml are sufficient to sustain the proliferation of NK cells. Thus, it can be anticipated that low dose IL2 (typically lower than 1 million units/square meters/day for daily subcutaneous injection) will be sufficient to sustain proliferation. In vitro, CD25 down regulated after 9-10 days, so that it is anticipated that the length of the low dose IL2 treatment will be up to 10 days.

1-15. (canceled)

16. A composition comprising an anti-NCR antibody and a cytokine, in association with a pharmaceutically acceptable carrier, said antibody(ies) and cytokine(s) being administered together or separately to a subject in an amount effective to stimulate the proliferation of NK cells.

17. The composition of claim 16, wherein said cytokine is an interleukin.

18. The composition of claim 17, wherein said interleukin is selected from the group consisting of IL2, IL12, IL15, IL21 and combinations thereof.

19. The composition of claim 16, wherein said antibody(ies) is an anti-NKp30 antibody or anti-NKp46 antibody, a combination of both anti-NKp30 antibody and anti-NKp46 antibody, or immunoreactive fragments thereof.

20. The composition of claim 19, wherein said antibody or antibodies are used in admixture with IL2.

21. The composition of claim 19, wherein said anti-NKp30 antibodies are isolated antibodies or antigen binding fragments thereof which specifically bind to a polypeptide selected from the group consisting of SEQ ID No: 1, SEQ ID No: 2, SEQ ID No: 3, SEQ ID No: 4, or an immunogenic fragment thereof, and SEQ ID No: 5.

22. The composition of claim 21, wherein said antibodies specifically bind to polypeptide comprising SEQ ID No: 1.

23. The composition of claim 19, wherein said anti-NKp30 antibodies, anti-NKp46 antibodies or combinations thereof are monoclonal antibodies, affinity, chimerized, humanized antibodies or antibodies of human origin.

24. The composition of claim 23, wherein said anti-NKp30 monoclonal antibody is produced by hybridoma strain 1-2576.

25. The composition of claim 16, wherein said NCR antibodies comprise antibody fragments, said fragments being essentially Fab, F(ab′)₂, and Fv fragments and CDR grafted humanized monoclonal antibodies.

26. The composition of claim 1, wherein said composition is in the form of tablet, powder, pastes, patches, granules, microgranules, nanoparticules, colloid solution, aqueous solution, injectable solutions, sprays, or liposomes.

27. The composition of claim 1, comprising from 1 ng to 100 mg/kg (body weight) of antibodies, and lower than 1 million units/square meters/day of cytokine(s).

28. A method for stimulating the proliferation of NK cells which comprises contacting NK cells with an effective amount of a pharmaceutical composition according to claim 16.

29. The method of claim 28, wherein one or several injections of an effective amount of said composition occurs for 5-10 days and said cytokine(s) being first injected on the same day as the first injection of antibodies.

30. The method of claim 29, comprising one or two injections/day of cytokine(s) by subcutaneous injection.

31. The method of claim 28, wherein said interleukin is IL-2 and is injected subcutaneously at daily doses below 1 million units/m² for 5 to 10 days.
